(19) United States
(12) Patent Application Publication
(10) Pub. No.: US 2006/0010004 A1
(43) Pub. Date: Jan. 12, 2006

(54) Method for providing customized products

(76) Inventor: George Endel Deckner, Cincinnati, OH (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 11/176,138

(22) Filed: Jul. 7, 2005

Related U.S. Application Data
(60) Provisional application No. 60/586,603, filed on Jul. 9, 2004.

(51) Int. Cl. G06Q 99/00; A61K 8/02

(52) U.S. Cl. 705/1; 424/401

(57) ABSTRACT

In a first aspect a method for producing a customized product is disclosed. The method includes providing a high internal phase emulsion, providing at least one other component of a customized product, generating a customized product formula based at least in part on consumer data and mixing the emulsion with the at least one other component in accordance with the customized product formula to produce the customized product. In a second aspect, a method of manufacturing a customized personal product using an oil-in-water high internal phase emulsion is disclosed.

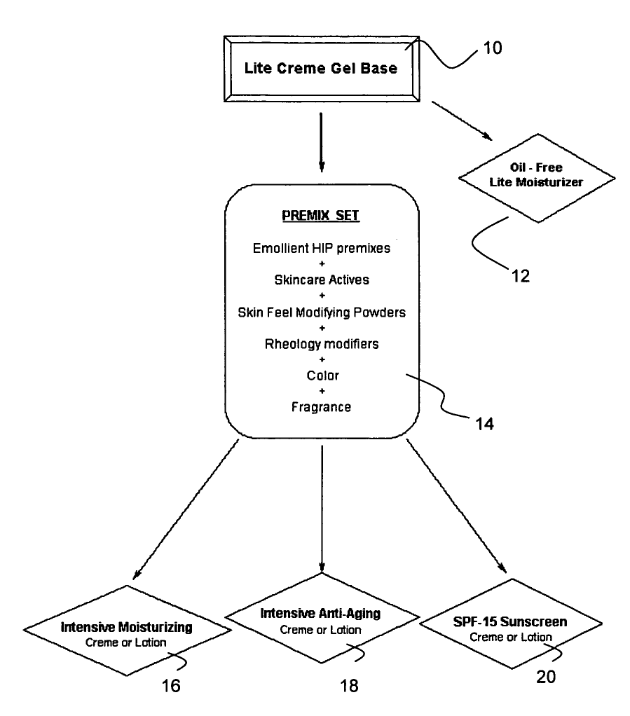

### CROSS REFERENCE TO RELATED APPLICATION

This Application claims the benefit of U.S. Provisional Application No. 60/586603, filed Jul. 9, 2004.

### FIELD OF THE INVENTION

The present invention relates to a method for providing customized products, such as personal care, healthcare or laundry products.

### BACKGROUND OF THE INVENTION

The majority of consumer products are mass produced with very little, if any, personalization of the product for the specific wants or needs of individual consumers. It is becoming increasingly desirable for a company to manufacture products which are more specifically tailored to the consumer's wants and needs. Existing systems for providing customized products to consumers typically require sophisticated mixing devices to ensure that the various components of the formulation are properly mixed to provide a uniform product which can be consistently produced at different times and locations.

Stabilized emulsions have been used in manufacturing operations to facilitate processing and formulation development. An emulsion is a dispersion of one liquid phase in another, substantially immiscible, continuous liquid phase. Water-in-oil (or oil in water) emulsions having a high ratio of dispersed phase to continuous phase are known in the art as High Internal Phase Emulsions (hereafter referred to as “HIPE” or HIPEs). HIPEs have found application in a number of technologies, such as fuels, cosmetics and foods—an everyday example of these emulsions is mayonnaise (which may typically comprise about 70% vegetable oil in water). These concentrated emulsions have also found application in the cosmetic area because the concentrates can stably contain high concentrations of, for example, emollients, moisturizers and sunscreens, which can then be diluted down using simple cold mixing to obtain the desired end product. Reference may be made to U.S. Pat. No. 4,606,913 and U.S. Pat. No. 5,976,604, which teach concentrated emulsions.

It would be beneficial to develop a system for use at the point of sale which allows the consumer to customize the characteristics of the product. In addition, it would be advantageous to utilize HIPEs in the system to provide excellent quality and consistency of the finished product and yet allow for a variety of characteristics to be selected by the consumer to provide a personalized product. In particular, it would be desirable to provide a method of producing a custom personal care, healthcare or laundry product at the point of sale using an oil-in-water HIPE base.

### SUMMARY OF THE INVENTION

The present invention relates to a method for providing a consumer with customized products, such as personal care, healthcare or laundry products. More particularly, the present invention in accordance with certain embodiments relates to a method for providing customized products at the point of sale using high internal phase emulsions mixed with other components.

According to a first aspect of the invention, a method for providing a customized product, preferably to a consumer at the point of sale, is disclosed. In accordance with this aspect of the invention, an emulsion is provided including a discontinuous phase, a continuous phase and emulsifier, wherein the emulsion comprises at least 50% by weight of the discontinuous phase, at least one other component is provided, a customized product formula is generated based at least in part on consumer data and the emulsion is mixed with the at least one other component in accordance with the customized product formula to produce the customized product for the consumer.

According to a second aspect of the invention, a method of manufacturing a customized personal care product is disclosed. In accordance with this aspect of the invention, a concentrated emulsion is provided including at least 50% by weight of the emulsion of a discontinuous oil phase, a continuous aqueous phase and an emulsifier, a plurality of compositions are provided, each composition including at least one other component of the customized product, a customized product formula is generated based at least in part on consumer data and the emulsion is mixed with at least one of the compositions in accordance with the customized product formula.

### BRIEF DESCRIPTION OF THE DRAWING

The accompanying drawing, incorporated in and forming part of the specification, illustrates one aspect of the present invention and, together with its their description, serves to explain the principles of the invention. In the drawings:

The FIGURE depicts a flow diagram for a method of providing a customized lotion to the consumer.

### DETAILED DESCRIPTION OF THE INVENTION

All weights, measurements and concentrations herein are measured at 25° C. on the composition in its entirety, unless otherwise specified.

Unless otherwise indicated, all percentages of compositions referred to herein are weight percentages of the total composition (i.e. the sum of all components present) and all ratios are weight ratios.

Unless otherwise indicated, all polymer molecular weights are weight average molecular weights.

Unless otherwise indicated, the content of all literature sources referred to within this text are incorporated herein in full by reference.

Except where specific examples of actual measured values are presented, numerical values referred to herein should be considered to be qualified by the word “about”.

As used herein, the term “oil-in-water” or “o/w” means that an oil phase is dispersed in an aqueous phase, such that the aqueous phase is the continuous phase and the oil phase the discontinuous phase.

As used herein, the term “water-in-oil” or “w/o” means that an aqueous phase is dispersed in an oil phase, such that the oil phase is the continuous phase and the aqueous phase is the discontinuous phase.

The present invention provides a method for producing a customized product based at least in part on consumer data. Consumer data may be data provided by the consumer or data derived from consumer studies. In accordance with particular embodiments of the present invention, the customized product is produced at the point of sale. In accordance with alternative embodiments of the invention, the customized product can be produced at a remote location in response to customer data supplied by mail order, phone order or internet purchase.

In accordance with certain embodiments, a consumer may input these selection data in a variety of ways such as with a color slide selector, keyboard, mouse, computing device, digital phone, digital camera, digital video recorder, and the like. These selections made by the consumer may be retrieved from a computer readable medium or these selections may be saved from previously made selections and stored to a computer readable medium. Once the consumer has determined the selections for his/her customized cosmetic product, the proportions of the base ingredients will be known. Calculating the proportions of known base ingredients necessary to create a specific type (product selection) of cosmetic product is well known and readily ascertainable to those skilled in the art. Moreover, creating the appropriate color shading of a cosmetic or other product is trivial, once the consumer has made his/her color selections. The appropriate ingredient proportions are calculated and the ingredients are then combined to form the desired product.

The present invention provides a method for producing customized products including, but not limited to, health care products, personal care products and laundry products. The present invention will be further illustrated by reference to methods for producing customized lotions. In accordance with this embodiment of the present invention a high internal phase emulsion is mixed with at least one other component of a customized lotion product wherein the customized product formula is based at least in part on data provided by a consumer.

The FIGURE depicts a flow diagram for a method of providing a customized lotion to the consumer. The base composition would vary depending upon the type of product being customized. In this case, a light creme gel base 10 is used to prepare customized lotions. Light creme gel base 10 can be used to produce a product identified as oil-free light moisturizer 12 directly without adding any optional customization ingredients. Customized lotions or cremes can be produced based on incorporating various optional ingredients 14 into the light creme gel base 10. The classes of variable components include, but are not limited to, emollient HIP premixes, skin care actives, skin feel modifying powders, rheology modifiers, color and fragrance. Non-limiting examples of various options within these classes are set forth below.
Moisturizing actives such as petrolatum, glycerin, lanolin
Anti-aging actives such as panthenol (pro vitamin B5) niacinamide, tocopheryl acetate (vitamin E), Pitera, peptides (Matrixyl), MMP inhibitors (MDI Complex), Ursolic acid
Botanical actives (Floraceuticals containing a certified level of plant actives)
Fragrances or unscented
Colors: white, peach, or pink
Consistency: from fluid lotion to thick cream
Feel modifying agents such as powders, waxes and liquid emollients.
By varying the available optional ingredients a consumer can create virtually any sensory profile desired.

In accordance with the described customization process, the consumer selects a general type of lotion such as intensive moisturizing 16, intensive anti-aging 18 or SPF-15 sunscreen 20. The consumer's selection of the general type of creme or lotion will define some basic properties of the composition such as consistency and the types of optional ingredients available for further modification. For example, a consumer selecting SPF-15 sunscreen 20 may be able to further modify the sunscreen to provide a different SPF, fragrance and color.

The consumer data can be used to generate a customized product formula. The customized product formula can be printed out and the various components manually mixed or the entire operation can be conducted automatically using standard mechanical pumps, mixing devices, dispensers, microprocessors and ingredients well known in the art. Various methods for collecting data from the consumer are contemplated including the techniques described in U.S. Patent Application Publication No. 2003/0014324 A1, Donovan et al., published Jan. 16, 2003 (assigned to the Procter & Gamble Company). For example, the consumer may provide responses to a series of questions which are either directly or indirectly related to the specific customized product. In accordance with one embodiment, the data used to customize the product is collected through a user interface in which the consumer is presented questions which elicit the necessary data. The collection of the data may also be obtained through mail surveys, test kits, in-person consultation or direct interaction. In accordance with another embodiment, the data may be collected by measuring a physical attribute of the consumer. For example, the consumer may be provided with a test kit which determines conditions such as skin dryness, skin oiliness, hair dryness, wrinkling and color complexion of the consumer. Alternatively, a device such as a spectrophotometer or colorimeter could be used to generate data characterizing an attribute of the consumer.

Preferably, the process of collecting the data input from the consumer and determining the proper formula occur in a microprocessor environment wherein interfacing with the consumer and performing the ingredient calculations are simple tasks. The microprocessor environment makes software and hardware readily available and easily ascertainable to those skilled in the art to perform these steps.

The finished customized formulation may be dispensed directly to the consumer for immediate use or it may be packaged and used at a later time. Furthermore, the customized formulation could be packaged and sent to the consumer at a remote location.

Many of the advantages associated with the present invention derive from the use of high internal phase emulsions which allow the final customized product to be produced using only low shear mixing. The high internal phase emulsion and the other components of the customized product can be either hand stirred or mechanically mixed to provide a uniform and consistent product. Furthermore, the compositions of the present invention can be mixed at or near ambient temperatures and, therefore, are suitable for use with heat liable actives and fragrances. Although the high internal phase emulsions can be either oil-in-water or water-in-oil, the oil-in-water emulsions are preferred and the remaining discussion will focus on the use of these emulsions. Oil-in-water emulsions typically will be mixed with other aqueous pre-rmixes to produce the finished formulation. In accordance with a particularly useful embodiment of the invention, the emulsions are emulsified by a non-alkoxylated water-soluble emulsification polymer.

In accordance with particular embodiments of the present invention, the products are formulated based on emulsions having a concentrated internal or discontinuous oil phase, which represents at least 50% by weight of the emulsion, preferably at least 70%, more preferably at least 80% and more preferably still from 80 to 93% by weight of the emulsion.

As used herein, the term “non-alkoxylated” in relation to the water-soluble emulsification polymers means polymers comprising no alkoxy groups, that is no —OR groups (where R includes alkyl moieties) in the molecule, neither in the polymer backbone, nor as pendants thereto nor elsewhere.

The oil phase according to this aspect of the invention may comprise any water immiscible material that is liquid at ambient conditions; any material that is solid at ambient conditions, has a melting temperature of less than 100° C. and melts to form a water immiscible liquid; mixtures of such materials.

As used herein in relation to the oil phase, the term “water immiscible” includes materials having a Hildebrand Solubility Parameter of around 5-12 calories/cc (209-502 kJ/m²). The solubility parameter is defined as the sum of all attractive forces radiating out of a molecule. The total Van der Waals force is called the Hildebrand Solubility Parameter and can be calculated using Hildebrand's equation using boiling point and MW data. Methods and a computer program for calculating the Hildebrand Solubility Parameter are disclosed by C. D. Vaughan in J. Cosmet. Chem. 36, 319-333 (September/October 1985).

Materials comprised within the oil phase may have any polarity and may include aliphatic or aromatic hydrocarbons, esters, alcohols, ethers, carbonates, fluorocarbons, silicones, fluorosilicones or derivatives thereof.

Solid materials that may be present in the oil phase include waxes. As used herein, the term “wax” includes natural and synthetic waxes. The class of natural waxes includes animal waxes, such as beeswax, lanolin, shellac wax and Chinese insect wax; vegetable waxes, such as carnauba, candelilla, bayberry and sugar cane; mineral waxes, such as ceresin and ozokerite; petrochemical waxes, such as microcrystalline wax and petrolatum. The class of synthetic waxes includes ethylenic polymers and polyol ether-esters, chlorinated naphthalenes and Fischer-Tropsch waxes. For more details, please refer to see Römpp Chemie Lexikon, Georg Thieme Verlag, Stuttgart, 9^{th} Edition, 1995 under “Wachse”.

Advantageously, materials comprised within the oil phase, including the melted waxes, have a viscosity in the range from 0.005 to 30,000 cm²/s (0.5 to 3,000,000 cst), preferably from 0.005 to 20,000 cm²/s (0.5 to 2,000,000 cst), more preferably from 0.005 to 3500 cm²/s (0.5 to 350,000 cst).

The aqueous phase of the emulsions according to this aspect of the invention comprises water and may also comprise additional water-soluble components, such as alcohols; humectants, including polyhydric alcohols (e.g. glycerine and propylene glycol); active agents such as d-panthenol, vitamin B₃ and its derivatives (such as niacinamide) and botanical extracts; thickeners and preservatives.

Advantageously, the emulsions according to the invention comprise substantially no electrolyte. As used herein, the term “electrolyte” includes substances that form ions in aqueous solution and the term “substantially no electrolyte” means that an emulsion according to the invention comprises less than 0.001% electrolyte by weight of the emulsion. It is beneficial to exclude electrolytes because the diluted emulsions may become much more difficult to thicken when they are present—many thickeners are highly sensitive to salt levels. Thickeners are frequently used in emulsions to increase the viscosity of the water phase, thereby reducing the ability of oil phase droplets, which typically have a lower density than the aqueous phase, to rise to the top of an emulsion formulation (so-called “creaming”).

Preferably, the viscosity of the aqueous phase does not exceed 2 kg/ms (2000 cps), measured using a Brookfield Digital Rheometer Model DV-III, with an RV2 spindle at 20 rpm (Brookfield Engineering Laboratories—Stoughton Mass.) at 20° C. Above this point, emulsification may become extremely difficult, especially when the internal oil phase is present at high levels, such as 80-90% by weight of the emulsion.

The water-soluble emulsification polymers according to this aspect of the invention have a molecular weight of at least 500 Daltons, since below this level, the resulting emulsions have poor skin feel. Skin feel improves with increasing molecular weight and it is preferred that the water-soluble emulsification polymers according to the invention have a molecular weight above 3000 Daltons, more preferably above 9000 Daltons and more preferably still, above 10,000 Daltons.

At the levels of emulsifier present in the emulsions according to certain aspects of the invention, the molecular weight of the emulsification polymers advantageously does not exceed 130 kiloDaltons; above this point, the viscosity of the aqueous phase may reach a level that hinders emulsification, especially when the internal oil phase is present at levels of 80-90% by weight of the emulsion.

Advantageously, from 70% to 100% of the total weight of emulsifier comprised within the present emulsions contains one or more non-alkoxylated water-soluble emulsification polymers.

Surprisingly, it has been found that any non-alkoxylated, water-soluble polymer fulfilling the defined molecular weight and surface tension criteria may be used to emulsify the above-described emulsions and are capable of mitigating the problems encountered in the prior art. This applies regardless of the chemical nature of the water-soluble polymer, so that polymers of widely differing chemistries may be employed. Non-limiting water-soluble polymers which may be employed according to the invention include: alkylated polyvinylpyrrolidone, such as buylated polyvinylpyrrolidone commercialised as “Ganex P904” by ISP Corp.; mono alkyl esters of poly(methyl vinyl ether/maleic acid) sodium salt, including mono butyl ester of poly(methyl vinyl maleic acid sodium salt) such as included in the product commercialised as “EZ Sperse” by ISP Corp; isobutylene/ethylmaleimide/hydroxyethyl copolymer, such as included in the product commercialised as “Aquafix FX64” by ISP Corp.; (3-dimethylaminopropyl)-methacrylamide/3-methacryloylamidopropyl-lauryl-dimthyl-ammonium chloride, such as included in the product commercialised as Styleze W20 by ISP Corp.

Advantageously, at least one of the non-alkoxylated, water-soluble polymers according to certain aspects of the invention has film-forming properties. These properties are found in higher molecular weight polymers, especially those having a molecular weight above 10,000 Daltons. The film-forming property may further increase the substantivity of the emulsions on the substrate versus traditional surfactants, including alkoxylated surfactants. Dried-down oil-in-water emulsions comprising traditional surfactants, including alkoxylated surfactants, suffer from the disadvantage that they may re-emulsify when wetted, whereas the present non-alkoxylated, water-soluble polymers are less liable to do that. Without wishing to be bound by theory, it is believed that the substantivity of the present compositions may be further increased if the polymers exhibit film-forming properties, because the film-forming polymer may form a film over the oil phase to retain it on the substrate.

The emulsions according to this aspect of the invention may comprise from 0.1% to 15%, preferably from 0.1% to 5% and more preferably 0.1 to 2.5% by weight water-soluble emulsification polymer.

The emulsions according to this aspect of the invention may be manufactured in the following way:

A typical emulsion might contain 1-5% water-soluble emulsification polymer and 8-9% aqueous phase, the aqueous phase comprising 100% water or a mixture of water and other water-soluble components. In a first step, the water-soluble emulsification polymer is added to the aqueous phase with mixing. Following this, discrete batches of 6-15% of the total weight of oil are titrated sequentially into the aqueous phase accompanied by gentle mixing to obtain a uniform consistency prior to addition of the following batch. This is continued until around 20% of the total weight of oil has been added. At this point the remainder of the oil may be added more rapidly and in a continuous fashion with more vigorous mixing until a uniform emulsion comprising all the oil is obtained. Mixing is continued until a uniform consistency is obtained exhibiting a typical particle in a desired range. A typical particle size would be in the range from 1 to 20 microns. The concentrated emulsion obtained typically comprises above 70%, and more often from 80 to 93% internal oil phase by weight of the emulsion.

The HIPEs in accordance with the present invention are used to produce customized products based on consumer data, preferably obtained from a consumer in a retail point of sale environment. Examples of such products include personal care products, such as lotions for hand and body, shampoo compositions, make-up, perfume and perfume gel compositions and lotions for baby wipes; laundry products such as fabric softener and liquid laundry detergent; health care products, such as vapour rub creams; coatings for tissue towels.

Personal care, health care and laundry products may comprise from 0.01 to 30% wt, preferably from 0.25 to 12% wt, more preferably 0.25 to 5% wt of the above-defined concentrated emulsions. Accordingly, other components of the products may be present at amounts ranging from 99.99 to 70%, preferably from 99.75 to 88% and more specifically from 99.75 to 95%.

Non-limiting examples of materials that may be included in such products are thickeners; surfactants, such as non-ionic, anionic, cationic, zwitterionic and amphoteric surfactants; humectants, such as polyhydric alcohols, including glycerine and propylene glycol; pigments, including organic and inorganic pigments; preservatives; chelating agents, antimicrobials, perfumes. In accordance with particular embodiments, materials mixed with the emulsions are in the form of aqueous pre-mixes containing one or more components of the finished formulation.

Customized products in accordance with certain aspects of the present invention are prepared from high internal phase emulsions and at least one aqueous pre-mix wherein the high internal phase emulsion is produced in a first location, the at least one aqueous pre-mix is produced in the same location or in a second location and then the high internal phase emulsion and at least one aqueous premix are transported to a third location for use in manufacturing the customized product. In particular embodiments, the third location is a retail store and the finished product is manufactured on-site in the store.

### EXAMPLE

The following example further describes and demonstrates a particular embodiment of the present invention. The example is given solely for the purpose of illustration, and is not to be construed as limitations of the present invention since many variations thereof are possible without departing from its scope.

A customized lotion is produced by adding optional ingredients to a Gel Base, the components of which are set forth below:

Lite Moisturizer Crème/Gel Base

Water
Glycerin
Hexylene Glycol
Dimethicone and Dimethiconol (DC 2-1503)
Caprylic/Capric Triglyceride (and) Sodium Acrylates Copolymer
(Luvigel EM)
Cetaryl Alcohol (and) Hydrogenated polyisobutene (AM 900)
Benzyl Alcohol
Ethyl Paraben
Decyl glucoside
Laureth 4
Titanium Dioxide

The gel base is modified by adding one or more of the following optional ingredients based on data provided by a consumer to produce the customized lotion.

Customization Menu

| Colors | Moisturizing Ingredients |
|---|---|
| White | Glycerin |
| Peach | Petrolatum |
| Pink | Lanolin |
| Fragrances | Rheology Modifiers |
| Olantra | Luvigel EM |
| Eau Mod 3 | Novomer EC-1 |
| New York Martini | Sepigel 305 |
| Glamorous | Salt |
| Unscented | |
| Actives | Feel Modifying Powders |
| Pitera 4X | Nylon |
| Panthenol | PTFE (FluoroPure 100) |
| Niacinamide | Silicone Resin (Tospearl) |
| Vitamin E | Modified starch (Dry Flow) |
| Ginseng | |
| Peptide (Matrixyl) | Emollients |
| Ginko Biloba | Cetyl/stearyl alcohol |
| Green Tea | Silicone gum blend |
| Chamomile | PETIS |
| Centella | Silicone elastomer gel |
| Purple Coneflower | Hyd polyisobutene |
| | SEFA |
| | Isohexadecane |
| | Isopropyl isostearate |

The calculated amounts of the selected ingredients according to the customized product formula are mixed by hand or using a small mixer to produce a uniform customized lotion. The lotion can be dispensed directly to the consumer or packaged and delivered to the consumer at a remote location.

It is understood that the examples and embodiments described herein are for illustrative purposes only and that various modifications or changes in light thereof will be suggested to one skilled in the art without departing from the scope of the present invention.

All documents cited in the Detailed Description of the Invention are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method for producing a customized product comprising the steps of:
a) providing an emulsion comprising a discontinuous phase, a continuous phase and emulsifier, wherein said emulsion comprises at least 50% by weight of the discontinuous phase,
b) providing at least one other component of a customized product;
c) generating a customized product formula based at least in part on consumer data; and
d) mixing the emulsion with the at least one other component in accordance with the customized product formula to produce the customized product for the consumer.

2. The method according to claim 1, wherein the emulsion is a water-in-oil emulsion.

3. The method according to claim 1, wherein the emulsion is an oil-in-water emulsion.

4. The method according to claim 3 wherein the emulsifier comprises a non-alkoxylated water-soluble emulsification polymer having a molecular weight of at least 500 Daltons, a 0.1% wt aqueous solution of the water-soluble emulsification polymer having a surface tension of 15-60 mN/m (15-60 dynes/cm) when measured at 25° C.

5. The method according to claim 4 wherein the emulsion comprises at least 70% by weight of the discontinuous phase.

6. The method according to claim 1 further comprising:
e) dispensing the customized product to a consumer at a retail point-of-sale.

7. The method according to claim 1 wherein the customized product is selected from the group consisting of a healthcare product, a personal care product and a laundry product.

8. The method of claim 7 wherein the customized product is a personal care product selected from the group consisting of a fragrance, a cosmetic product, a lotion, a shampoo, a make-up, a perfume gel, a skin care product and a hair care product.

9. The method according to claim 1 wherein step d comprises low shear mixing at approximately ambient temperature.

10. The method of claim 1 wherein the component comprises an ingredient selected from the group consisting of an active ingredient, a color, a fragrance, a moisturizing ingredient, a rheology modifier, a modifying powder and mixtures thereof.

11. The method of claim 1 wherein the consumer data comprises data provided by a consumer at a retail point of sale.

12. The method of claim 11 wherein the data provided by the consumer comprises responses by the consumer to a series of questions.

13. The method of claim 12 wherein the data provided by the consumer comprises data characterizing at least one physical attribute of the consumer.

14. The method of claim 1 wherein the the emulsion is present in the customized product at a content of from about 0.01 to 30% wt.

15. The method of claim 2 wherein said at least one other component comprises an aqueous pre-mix.

16. A method of manufacturing a customized personal care product comprising the steps of:
a) providing a concentrated emulsion comprising at least 50% by weight of the emulsion of a discontinuous oil phase, a continuous aqueous phase and an emulsifier,
b) providing a plurality of compositions each composition comprising at least one other component capable of being used in a customized product;
c) generating a customized product formula based at least in part on data provided by a consumer; and
d) mixing the emulsion with at least one of the compositions in accordance with the customized product formula.

17. The method according to claim 16 wherein the emulsifier comprises a non-alkoxylated water-soluble emulsification polymer having a molecular weight of at least 500 Daltons, a 0.1% wt aqueous solution of the water-soluble emulsification polymer having a surface tension of 15-60 mN/m (15-60 dynes/cm) when measured at 25° C.

18. The method according to claim 16 wherein the emulsion comprises at least 70% by weight of the discontinuous phase.

19. The method according to claim 16 wherein the oil phase particle size is in the range from about 1 to 20 μm.

20. The method according to claim 16 wherein concentrated emulsion comprises from about 0.01 to 30% wt of the personal care composition.

21. The method of claim 16 wherein the personal care product is selected from the group consisting of a fragrance, a cosmetic product, a lotion, a shampoo, a make-up, a perfume gel, a skin care product and a hair care product.

22. The method according to claim 16 wherein step d comprises low shear mixing at approximately ambient temperature.

23. The method of claim 16 wherein the component is selected from the group consisting of an active ingredient, a color, a fragrance, a moisturizing ingredient, a rheology modifier, a modifying powder and mixtures thereof.

24. The method of claim 23 wherein said component comprises an aqueous pre-mix.

25. The method of claim 22 wherein step d occurs at a retail point of sale.

26. A method for producing a customized product comprising the steps of:
a) preparing an emulsion comprising a discontinuous phase, a continuous phase and an emulsifier, wherein said emulsion comprises at least 50% by weight of the discontinuous phase,
b) preparing an aqueous pre-mix comprising at least one other component capable of being used in a customized product,
c) generating a customized product formula based at least in part on data provided by a consumer; and
d) mixing the emulsion with the aqueous pre-mix in accordance with the customized product formula to produce the customized product, wherein said emulsion is prepared in a first location, said aqueous pre-mix is prepared in said first location or in a second location and step d occurs in a third location.

27. The method according to claim 26 wherein the emulsion is an oil-in-water emulsion.

28. The method according to claim 26 wherein the emulsifier comprises a non-alkoxylated water-soluble emulsification polymer having a molecular weight of at least 500 Daltons, a 0.1% wt aqueous solution of the water-soluble emulsification polymer having a surface tension of 15-60 mN/m (15-60 dynes/cm) when measured at 25° C.

29. The method according to claim 28 wherein the emulsion comprises at least 70% by weight of the discontinuous phase.

30. The method according to claim 26 wherein the customized product is selected from the group consisting of a healthcare product, a personal care product and a laundry product.

31. The method of claim 30 wherein the customized product is a personal care product selected from the group consisting of a fragrance, a cosmetic product, a lotion, a shampoo, a make-up, a perfume gel, a skin care product and a hair care product.

32. The method of claim 26 wherein the component comprises an ingredient selected from the group consisting of an active ingredient, a color, a fragrance, a moisturizing ingredient, a rheology modifier, a modifying powder and mixtures thereof.

33. A system for retail point-of-sale production of a customized product comprising:
a) a concentrated emulsion comprising at least 50% by weight of the emulsion of a discontinuous oil phase, a continuous aqueous phase and an emulsifier wherein said emulsion is produced in a first location,
b) an aqueous pre-mix comprising at least one other component capable of being used in a customized product, wherein said aqueous pre-mix is produced in said first location or at a second location, and
wherein said emulsion and said aqueous pre-mix are transported to a third location for custom blending to produce a customized product having a formulation based at least in part on consumer data.
